(19) United States
(12) Patent Application Publication
(10) Pub. No.: US 2009/0136434 A1
(43) Pub. Date: May 28, 2009

(54) Tyrosinase enzyme inhibitors from fungal hydroxylation of tibolone

(75) Inventors: Attaur Rahman, Karachi (PK); Muhammad Iqbal Choudhary, Karachi (PK); Syed Adnan Ali Shah, Selangot (MY); Mahmud Tareq Hassan Khan, Chittagong (BD)

Correspondence Address: SARFARAZ K. NIAZI, 20 RIVERSIDE DRIVE, DEERFIELD, IL, 60015, US

(73) Assignee: HEJ Research Institute, Karachi (PK)

(21) Appl. No.: 11/944,476

(22) Filed: Nov. 23, 2007

(51) Int. Cl. A61K 8/63; C07J 7/00; A61Q 17/04; A23B 7/154; A23B 4/20; A61Q 1/02; C12P 33/00

(52) U.S. Cl. 424/59; 552/597; 435/52; 424/63; 426/268

(57) ABSTRACT

Seven new and one known metabolites of tibolone were obtained by incubation of tibolone with various fungi. Their structures were elucidated by means of a homo and heteronuclear 2D NMR and by HREI-MS techniques. The relative stereochemistry was deduced by 2D NOESY experiment. These metabolites of tibolone were strong inhibitors of tyrosine enzyme and thus useful in producing skin lightning and preserving fruits and vegetables.

### SUMMARY OF INVENTION

This invention comprises discovery of several novel metabolites of tibolone that were potent inhibitors of tyrosinase enzyme; consequently, this invention reports cosmetic compositions of these new metabolites and skin lightening agents and their use in preventing enzymatic browning of fruits, vegetables and marine food products. The new metabolites of tibolone were obtained by novel methods of fermentation with various fungi.

### DETAILS OF INVENTION

### Discovery of New Tyrosinase Inhibitors

Microbial transformation is an effective tool to synthesize many steroidal drugs with potential biological activities. Such studies are primarily useful in the generation of hydroxylated metabolites for drug toxicity studies. Fungi, bacteria and yeast have been utilized successfully as in vitro models to mimic and predict the metabolic fate of drugs and other xenobiotics in mammalian systems. Previously, many biotransformation studies on various 17α-ethynyl steroids had been carried out with various fungal and bacterial strains, which afforded hydroxylation at various positions.

Tibolone (17-hydroxy-7α-methyl-19-norpregn-5(10)-en-20-yn-3-one) is a synthetic steroid that combines estrogenic and progestogenic properties with androgenic property, which mimic the action of a male sex hormone. The in vivo metabolism of tibolone in human had been studied with the reference to its three metabolites, 3α-hydroxytibolone, 3β-hydroxy tibolone and Δ⁴-tibolone.

In this invention, tibolone was used as a structural probe to identify its metabolites produced through microbial model. These metabolic studies resulted in isolation and identification of novel hydroxylation at various positions and known hydroxylation such as the compound Δ⁴-tibolone, which is a known metabolite in humans. It is well established that the nature of microbial biotransformation is unpredictable and dependent, to a great degree on the organism used, the substrate used, the conditions of fermentation used and thus it is not possible to predict the novelty of compounds thus obtained.

Tibolone when incubated with Rhizopus stolonifer, Fusarium lini, Cunninghamella elegans and Gibherella fujikuroi, resulted in the formation of several hydroxyl derivatives:

### Metabolite 1: 6β-Hydroxytibolone, MF C₂₁H₂₈O₃

### Metabolite 2: 15β-Hydroxytibolone, MF C₂₁H₂₈O₃

### Metabolite 3: Δ⁴-Tibolone C₂₁H₂₈O₂

### Metabolite 4: Δ^{1,4}-Tibolone C₂₁H₂₈O₃

Metabolite 5: 11α,15β-Dihydroxytibolone. C₂₁H₂₈O₄
Metabolite 6: 11α,15β-Dihydroxy-Δ⁵-tibolone C₂₁H₂₈O₄
Metabolite 7: 6β-Hydroxy-Δ⁴-tibolone C₂₁H₂₈O₃
Metabolite 8: 6β-Methoxy-Δ⁴-tibolone C₂₂H₃₀O₃

Tibolone is used effectively for the treatment of menopausal symptoms and in the prevention of osteoporosis, as a hormone replacement therapy (HRT). The hormone replacement therapy (HRT) effects on glucose metabolism in non-diabetic obese postmenopausal women. Tyrosinase (EC 1.14.18.1) is a multifunctional, copper-containing enzyme widely distributed in plants and animals. Tyrosinase inhibitors are clinically useful for the treatment of some dermatological disorders associated with melanin hyper pigmentation.

Here we are report a new class of tyrosinase inhibitors i.e., 17α-ethynyl steroids and compare their activity against the standard inhibitors of this enzymes and as a result report discovery of novel agents in the use of cosmetic care as well in the prevention of enzymatic browning of food products.

### EXPERIMENTAL

### General

Melting points were determined on a Yanaco MP-S3 apparatus. UV spectra were measured on a Shimadzu UV 240 spectrophotometer. IR spectra were recorded on a JASCO A-302 spectrophotometer in CHCl₃. ¹H- and ¹³C-NMR spectra were recorded on a Bruker Avance AM-400 spectrometer with tetramethylsilane (TMS) as an internal standard 2D NMR spectra were recorded on a Bruker Avance AMX 500 NMR spectrometer. Optical rotations were measured on JASCO DIP-360 digital polarimeter by using 10 cm cell tube. Mass spectra (EI and HREI-MS) were measured in an electron impact mode on Varian MAT 12 or MAT 312 spectrometers and ions are given in m/z (%). TLC was performed on a pre-coated silica gel card (E. Merck), spots were viewed with ultraviolet light at 254 nm for fluorescence quenching spots and at 366 nm for fluorescent spot and stained by spraying with a solution of eerie sulphate in 10% H₂SO₄. For column chromatography, silica gel (E. Merck, 230-400 mesh). Tibolone was extracted using dichloromethane.

### Fungi and Culture Conditions

Microbial cultures of the Fusarium lini (NRRL 68751), Rhizopus stolonifer (TSY 0471), Cunninghamella elegans (TSY 0865) and Gibberella fujikuroi were obtained from commercial sources widely available. These microbial cultures were grown on Sabouraud-4% glucose-agar (Merck) at 25° C. and stored at 4° C. Rhizopus stolonifer (TSY 0471) medium was prepared by adding glucose (100 g), peptone (25 g), KH₂PO₄ (25 g) and yeast extract (15 g) into distilled water (4 L) and pH was maintained at 5.6. Fusarium lini (NRRL 68751) and Cunninghamella elegans (TSY 0865) media were prepared by mixing the following ingredients into distilled H₂O (3.0 L) in each case: glucose (30.0 g), glycerol (30.0 g), peptone (15.0 g), yeast extract (15.0 g), KH₂PO₄ (15.0 g), and NaCl (15.0 g). Gibberella fujikuroi medium was prepared by adding the following ingredients into distilled H₂O (3.0 L): glucose (80.0 g), KH₂PO₄ (5.0 g), MgSO₄.2H₂O (1.0 g), NH₄NO₃ (0.5 g) and Gibberella trace element solution (2 mL). The Gibberella trace element solution was prepared by mixing Co(NO₃)₂.6H₂O (0.01 g), FeSO₄.7H₂O (0.1 g), CuSO₄.5H₂O (0.1 g), ZnSO₄.7H₂O (0.161 g), MnSO₄.4H₂O (0.01 g) and NH₄ molybdate (0.01 g) into distilled water (100 mL).

### General Fermentation and Extraction Conditions

The fungal media were transferred into 250 mL conical flasks (100 mL each) and autoclaved at 121° C. Seed flasks were prepared from three-day old slant and fermentation was allowed for two days on a shaker at 25° C. The remaining flasks were inoculated from seed flasks. After two days, tibolone was dissolved in acetone and transferred in each flask (15 mg/0.5 mL) and the flasks were placed on a rotary shaker (128 rpm) at 22° C. for fermentation period. The time course study was carried out after two days and the transformation was analyzed on TLC. The culture media were filtrated and extracted with CH₂Cl₂. The extract was dried over anhydrous Na₂SO₄, evaporated under reduced pressure and the brown gummy crude was analyzed by thin layer chromatography.

Fermentation of Tibolone with Rhizopus stolonifer (TSY-0471)

Tibolone (500 mg) was dissolved in 15 mL acetone and distributed among 40 flasks and allowed them for fermentation process. All the media were filtered after 3 days and extracted with dichloromethane and evaporated under reduced pressure to finally yield brown thick crude (0.90 mg), and the transformed metabolites were isolated by using column chromatography. Metabolite 1 (20 mg) was eluted with petroleum ether and ethyl acetate (60:40), metabolite 2 (17 mg) with petroleum ether and ethyl acetate (58:42) and metabolite 3 (40 mg) with petroleum ether and ethyl acetate (55:45). The following novel compounds were isolated and identified:

### 6β-Hydroxytibolone (Metabolite 1)

White amorphous solid (8.2 mg); nip 186-188° C.; [α]²⁵_{D}−17 (c 0.35, CHCl₃); UV (Methanol) λₘₐₓ(log ε) 204 (3.7) nm; IR (CHCl₃) νₘₐₓ3381, 2150, 1705, 1668, 1043 cm⁻¹; ¹H and ¹³C NMR data in CDCl₃, Tables 1 and 3; EIMS m/z (rel. int. %) 328 (M⁺, 6), 309 (5), 241 (16), 226 (9), 169 (14), 149 (23), 138 (28), 121 (28), 109 (23), 107 (100), 97 (20), 93 (21), 81 (26), 71 (22), 69 (41), 55 (64); HREIMS m/z 328.2171 (calculated for C₂₁H₂₈O₃, 328.2143).

### 15β-Hydroxytibolone (Metabolite 2)

White solid (7.6 mg); mp 202-205° C.; [α]²⁵_{D}+16 (c 0.31, CHCl₃); UV (Methanol) λₘₐₓ (log ε) 203.4 (3.4) nm; IR (CHCl₃) νₘₐₓ3383, 2162, 1708, 1663, 1050 cm⁻¹; ¹H and ¹³C NMR data in CDCl₃, Tables 1 and 3; EIMS m/z (rel. int. %) 328 (M⁺, 3), 312 (100), 245 (27), 229 (36), 203 (17), 189 (14), 187 (17), 174 (24), 161 (28), 149 (26), 135 (24), 121 (25), 96 (38), 81 (23), 69 (21), 67 (24), 55 (59); HREIMS m/z 328.2070 (calculated for C₂₁H₂₈O₃, 328.2038).

### Δ⁴-Tibolone (Metabolite 3)

Crystalline solid (20.4 mg); mp 206-208° C.; [α]²⁵_{D}−1.45 (c 0.21, CHCl₃); UV (Methanol) λₘₐₓ (log ε) 235 (3.2) nm; IR (CHCl₃) νₘₐₓ3402, 2150, 1687, 1667, 1017 cm⁻¹: ¹H NMR data in CDCl₃, Tables 1; ¹³C NMR (100 MHz, CDCl₃) δ 198.1 (C-3), 126.4 (C-4), 161.4 (C-5), 38.2 (C-10), 79.1 (C-17), 87.5 (C-20), 74.3 (C-21); EIMS m/z (rel. int. %) 312 (M⁺, 34), 245 (53), 229 (33), 187 (17), 173 (18), 161 (20), 147 (28), 135 (56), 121 (23), 109 (32), 107 (43), 105 (39), 95 (24), 91 (62), 81 (34), 79 (59), 67 (58), 55 (100); HREIMS m/z 312.2023 (calculated for C₂₁H₂₈O₂, 312.2089).

Fermentation of Tibolone by Fusarium lini (NRRL 68751) and Cunninghamella elegans (TSY 0865).

Tibolone (600 mg) was dissolved in 18 ml, acetone and distributed among 50 flasks, was kept for fermentation. Fermentation was continued for 6 days and then filtrates were extracted with dichloromethane and evaporated under reduced pressure to afford brown thick crude (1.02 gm). Column chromatography technique was used for the separation of metabolites from Cunninghamella elegans yielded one major metabolite 3. Compound 7 (6.2 mg) was eluted with petroleum ether and ethyl acetate (42:58), compound 4 (15.5 mg) with petroleum ether and ethyl acetate (38:62), 5 (5.2 mg) with petroleum ether and ethyl acetate (40:60), whereas metabolite 6 (10.2 mg) with petroleum ether and ethyl acetate (30:70), The following novel compounds were isolated and identified:

### 10β-Hydroxy-Δ⁴-Tibolone (Metabolite 4)

White powdered solid (15.5 mg); mp 198-201° C.; [α]²⁵_{D}+12 (c 0.25, CHCl₃); UV (Methanol) λₘₐₓ (log ε) 239 (2.9) nm; IR (CHCl₃) νₘₐₓ3345, 2149, 1698, 1649, 1018 cm⁻¹; ¹H and ¹³C NMR data in CDCl₃, Tables 1 and 3; EIMS m/z (rel. int. %) 328 (M⁺, 13), 310 (14), 229 (20), 187 (25), 171 (26), 161 (25), 149 (48), 136 (32), 124 (55), 109 (44), 107 (43), 91 (55), 83 (28), 67 (47), 57 (50), 55 (100); HREIMS m/z 328.2090 (calculated for C₂₁H₂₈O₃, 328.2123).

### 11α,15β-Dihydroxytibolone (Metabolite 5)

White powdered solid (5.2 mg); mp 208-210° C.; [α]²⁵_{D}−81 (c 0.24, CHCl₃); UV (Methanol) λₘₐₓ (log ε) 203 (3.3) nm; IR (CHCl₃) νₘₐₓ3342, 2142, 1718, 1636, 1028 cm⁻¹; ¹H and ¹³C NMR data in CDCl₃, Tables 1 and 3; EIMS m/z (rel. int. %) 344 (M⁺, 13), 310 (14), 229 (20), 187 (25), 171 (26), 161 (25), 149 (48), 136 (32), 124 (55), 109 (44), 107 (43), 91 (55), 83 (28), 67 (47), 57 (50), 55 (100); HREIMS m/z 344.2212 (calculated for C₂₁H₂₈O₄, 344.2234).

### 11α,15β-Dihydroxy-Δ⁵-Tibolone (Metabolite 6)

White powdered solid (7.3 mg); mp 207-211° C.; [α]²⁵_{D}+27 (c 0.28, CHCl₃); UV (Methanol) λₘₐₓ (log ε) 202.4 (3.4) nm; IR (CHCl₃) νₘₐₓ 3312, 2102, 1722, 1652, 1057 cm⁻¹; ¹H and ¹³C NMR data in CDCl₃, Tables 1 and 3; BUS m/z (rel. int. %) 344 (M⁺, 8), 310 (14), 229 (20), 187 (25), 171 (26), 161 (54), 149 (87), 136 (32), 124 (55), 109 (44), 1.07 (43), 91 (55), 83 (28), 67 (74), 57 (50), 55 (100); HREIMS m/z 344.2341 (calculated for C₂₁H₂₈O₄, 344.2316).

Fermentation of Tibolone by Gibberella fujikuroi (ATCC 10704)

Tibolone (850 mg) was dissolved in 20 mL acetone and distributed among 30 flasks for fermentation for 12 days. After fermentation, media was extracted with dichloromethane and evaporated to get a crude extract (1.22 gm). Column chromatography technique was used for the separation of metabolites 7 and 8 from crude extract. Metabolite 7 (10.2 mg) with petroleum ether and ethyl acetate (50:50), and metabolite 8 (10.3 mg) with petroleum ether and ethyl acetate (35:75). The following novel metabolites were isolated and identified:

### 6β-Hydroxy-Δ⁴-Tibolone (Metabolite 7)

White powdered solid (9.0 mg); mp 189-93° C.; [α]²⁵_{D}−101 (c 0.41, CHCl₃); UV (Methanol) λₘₐₓ (log ε) 239.5 (2.1) nm; IR (CHCl₃) νₘₐₓ 3332, 2128, 1698, 1651, 1063 cm⁻¹; ¹H and ¹³C NMR data in CDCl₃, Tables 1 and 3; EIMS m/z (rel. int. %) 328 (M⁺, 13), 312 (13), 245 (17), 229 (34), 189 (17), 187 (17), 161 (28), 149 (49), 121 (45), 91 (38), 69 (35), 67 (36), 55 (100); HREIMS m/z 328.2176 (calculated for C₂₁H₂₈O₃, 328.2116).

### 6β-Methoxy-Δ⁴-Tibolone (Metabolite 8)

White powdered solid (8.0 mg); mp 206-209° C.; [α]²⁵_{D}−18 (c 0.42, CHCl₃); UV (Methanol) λₘₐₓ (log ε) 242.1 (3.1) nm; IR (CHCl₃) νₘₐₓ 3323, 2153, 1691, 1661, 1101 cm⁻¹; ¹H and ¹³C NMR data in CDCl₃, Tables 2 and 4; EIMS m/z (rel. int. %) 343 (M⁺, 6), 312 (17), 245 (27), 229 (32), 203 (17), 189 (14), 187 (17), 174 (25), 161 (28), 149 (26), 135 (24), 121 (25), 91 (100), 81 (23), 69 (21), 67 (23), 55 (45); HREIMS m/z 343.2356 (calculated for C₂₂H₃₀O₃, 343.2338).

### RESULTS

Fermentation of Tibolone with Rhizopus stolonifer (TSY 0471) yielded two new mono-hydroxylated metabolites, 6β-Hydroxytibolone (Metabolite 1) and 15β-Hydroxytibolone (Metabolite 2), and a known metabolite Δ⁴-Tibolone (Metabolite 3). The HREIMS of Metabolite 1 exhibited the molecular ion (M⁺) at m/z 328.2171, corresponding to the formula C₂₁H₂₈O₃, which indicated that a new oxygen functionality was introduced into the molecule during fermentation period. The IR absorptions were attributed to hydroxyl (3381 cm⁻¹) and carbonyl (1705 cm⁻¹) functionalities, respectively. The ¹H NMR spectrum, compared with that of the substrate showed a new signal of OH-bearing methine proton at δ 4.04, resonating as a doublet (J=4.0 Hz) with its corresponding carbon resonating at δ 65.9 in ¹³C NMR spectrum which was assigned to C-6 on the basis of HMBC correlations of H-6 (δ 4.04) with C-5 (δ 122.5) and C-10 (δ 128.4). In the ¹H-¹H COSY 45° spectrum, the aforementioned methine proton showed correlation with the C-7 methine proton resonated at δ_{H} 2.0. The stereochemistry of C-6 hydroxyl group was determined to be axial by the NOESY correlations between H-6 (δ 4.04) and H-19 (δ 0.76). The above spectral data concluded that metabolite 1 has an —OH group at C-6 position as compared to tibolone and was deduced to be a new metabolite.

The HREIMS of metabolite 2 showed the M⁺ at m/z 328.2070, indicating an increment of 16 mass units as compared to tibolone in accordance to formula C₂₁H₂₈O₃. The ¹H and ¹³C NMR data of 5 revealed the presence of a new OH-bearing methine group that resonated at δ_{H} 4.06 (m, W_{1/2}˜10.8 Hz) and δ_{C} 65.5 and deduced for C-15 on the basis of HMBC spectrum correlations, which showed correlation of C-16 protons (δ_{H} 2.24, 1.7) and C-14 methine proton (δ_{H} 1.85) with C-15 (δ_{C} 65.5). The stereochemistry of the newly introduced C-15 hydroxyl group was deduced as β on the basis of NOESY correlations between H-15 (δ_{H} 4.06) and H-14 (δ_{H} 1.85) and multiplicity of H-15 signal at δ 4.06 (W_{1/2}˜10.8 Hz). From these spectral data, the new compound 5 was deduced to 7α-methyl-17α-ethynl-15β,17β-dihydroxy-19-norandrost-5(10)-en-3-one.

The incubation of tibolone with Fusarium lini (NRRL 68751) for 6 days also led to the isolation of a UV active metabolite 3 exhibiting the M⁺ at m/z 312.2023 in HREIMS spectrum (C₂₁H₂₈O₂). The ¹H NMR spectrum showed a singlet for an olefinic proton at δ 5.82. Its broad-band decoupled ¹³C NMR spectrum showed, in comparison with that of the substrate tibolone, the disappearance of one quaternary carbon signal resonating at. δ 128.2 for C-10 and appearance of an olefinic methine carbon at δ 126.4 which was assigned to the C-4 on the basis of HMQC spectrum, indicating the migration of the C-5/C-10 double bond to C-4/C-5. Thus creating an α,β-unsaturation in metabolite 3. The axial orientation of C-10 proton was assigned on the basis of NOESY coupling between H-10 (δ 2.31) and H-8 (δ 1.64). The above spectral data supported the structure of a known Metabolite 3 as 7α-methyl-17α-ethynl-17β-hydroxy-19-norandrost-4-en-3-one previously isolated during human metabolism of tibolone.

The incubation of tibolone (600 mg) with Cunninghamella elegans (TSY 0865) for six days yielded metabolites 4-6.

The HREIMS of metabolite 4 showed the M⁺ at m/z 328.2090, in agreement with the formula C₂₁H₂₈O₃ indicating an introduction of a new oxygen in the molecule, probably in the form of a hydroxyl group. However the ¹H NMR spectrum displayed no resonance for OH-bearing methine proton, but ¹³C NMR spectrum showed a downfield oxygen-bearing quaternary carbon resonated at δ 70.3, which was assigned to C-10 through its HMBC interactions with H-1 (δ 2.36, 2.29) and H-4 (δ 5.77). The 10β-hydroxylation was deduced by the β-SCS (substituents chemical shift) of −5.1, −5.4 and −6.9 ppm for C-2, C-8 and C-11, respectively, and by the downfield shifts of C-1 and C-9 (+5.1 and +7.5, respectively) with respect to the ¹³C NMR chemical shifts in compounds 1 and 4 (12). The spectral data supported the structure of a new metabolite 6 as 7α-methyl-17α-ethynl-10β,17β-dihydroxy-19-norandrost-4-en-3-one.

The HREIMS of metabolite 5 showed the M⁺ at m/z 344.2212 supporting the formula C₂₁H₂₈O₄, indicated that two oxygen had been incorporated into the molecule. The ¹H and ¹³C NMR displayed two OH-bearing methine groups resonating at δ_{H} 3.43 (ddd, J=15.1, 11.1, 5.0 Hz); and 4.1.0 (m, W_{1/2}˜8.82 Hz) and δ_{C} 66.1 and 65.4, respectively. The ¹H-¹H COSY 45° spectrum showed correlations of H-11 (δ 3.43) with H-9 (δ 1.62) and H₂-12 (δ 2.05, 1.51), and of H-15 (δ 4.10) with H-14 (δ 1.80) and H₂-16 (δ 2.01, 1.55). Hydroxylations at C-11 and C-15 was further supported by HMBC assignments, which has exhibited correlations of H₂-12 (δ 2.05, 1.51) and Me-18 (δ 0.90) with C-11 (δ 66.1), and correlation of H-14 (δ 1.80) with δ 65.4 (C-15). The axial orientation of C-11 proton was deduced on the basis of NOESY correlation of H-11 (δ 3.43) with Me-18 (δ 0.90) and multiplicity of H-11 signal resonating at δ_{H} 3.43 (ddd, J=15.1, 11.1, 5.0 Hz), ³ while (3-stereochemistry of the newly introduced OH group at C-15 was deduced by the NOESY correlations between H-14 (δ 1.80) and H-15 (δ 4.10) and multiplicity of H-15 signal resonating at δ 4.10 (W_{1/2}˜8.8 Hz). The p-orientation of C-10 proton was similar to metabolite 3. According to this spectral data, the structure was deduced to be 7α-methyl-17α-ethynl-11α,15β,17β-trihydroxy-19-norandrost-5(10)-en-3one.

The HREIMS of metabolite 6 showed the M⁺ at m/z 344.2341, support formula C₂₁H₂₈O₄, with an increment of 32 a.m.u. The UV spectrum showed a weak absorption at 202 nm, while IR showed absorptions at 3312 (OH), 1722 (C═O) and 1652 (C═C) cm⁻¹. The ¹H NMR spectrum showed an upfield doublet of olefinic methine proton at δ 5.42 (J=4.2 Hz, H-6), which showed COSY 45° correlations with H-7 (δ 1.83). Two additional OH-bearing methine protons resonating at δ 3.40 (ddd, J=15.3, 11.0, 4.57 Hz) and 3.91 (m, W_{1/2}˜9.9 Hz) were unambiguously assigned to H-11 and H-15 through 2D NMR and ¹³C NMR spectra. The stereochemistry of newly introduced hydroxyl group at C-11 was deduced to be α (equatorial) on the basis NOESY correlation between H-11 (δ 3.40) and H-18 (δ 0.94) and larger coupling constants (J=15.3 Hz) of H-11 signal.³ The β-orientation of the OH group at C-15 was deduced on the basis of NOESY correlation between H-14 (δ 1.85) and H-15 (δ 3.9.1) and multiplicity of H-15 signal, resonating at δ 3.91 (m, W_{1/2}˜9.9 Hz). The axial β-orientation of C-10 proton was deduced through NOESY cross peaks between H-10 (δ 2.46) and H-8 (δ 1.59) Based on the above mentioned spectral data, the structure was deduced as 7α-methyl-17α-ethynl-11α,15β,17β-trihydroxy-19-norandrost-5-en-3one.

Tibolone was fermented with Gibberella fujikuroi (ATCC 10704) for 12 days yielding six new mono-hydroxylated metabolites 7 AND 8. Metabolite 7 was found to be epimers and differentiated on the basis of ¹H NMR and NOESY experiments. The ¹H NMR spectrum of metabolite 7 displayed a doublet at δ 4.05 (J=4.0 Hz), while ¹³C NMR spectra of the isomer of metabolite 7 showed OH-bearing methine carbons resonating at δ 65.9 and 70.0, respectively. The position of the newly introduced hydroxyl at C-6 in both isomers was inferred from the HMBC coupling (12). The relative configuration in metabolite 7 of the new hydroxyl group at C-6 was inferred on the basis of coupling pattern and NOESY correlations between H-6 (δ 4.05) and C-19 methyl protons (δ 0.75). The above spectral data concluded that metabolite 7 has an —OH group at C-6 position with different orientations.

The HREIMS of metabolite 8 showed the M⁺ at m/z 343.2356 corresponding to the formula C₂₂H₃₀O₃. The ¹H NMR spectrum of metabolite 8 showed the presence of a methoxy singlet resonating at δ 3.47, while geminal methoxy protons were resonated at δ 3.93 (d, J=3.9 Hz). The ¹³C NMR spectrum showed a methoxy carbon signal at δ 57.6 and a methoxy-bearing carbon resonated at δ 70.2. The position of the newly introduced methoxy group at C-6 was deduced through HMBC interactions of H-6 (δ 3.93) with C-4 (δ 126.7). The β-orientation of the newly introduced OCH₃ group at C-6 was deduced on the basis of NOESY correlations between H-6 (δ 3.93) and C-19 methyl protons (δ 0.77).

The above mentioned spectral data led to conclude that, metabolite 8 has the structure as 7α-methyl-17α-ethynl-6β-methoxy-17β-hydroxy-19-norandrost-4-en-3-one.

### Tyrosinase Inhibition

### Material and Methods for Tyrosinase Inhibition Assay

Tyrosinase inhibition assays were performed in 96-well microplate format using SpectraMax 340 microplate reader (Molecular Devices, CA, USA) according to the method developed by Hearing (1987). Briefly, first the compounds were screened for the o-diphenolase inhibitory activity of tyrosinase using L-DOPA as substrate. All the active inhibitors from the preliminary screening were subjected to IC₅₀ studies. Compounds were dissolved in methanol to a concentration of 2.5%. 30 units of mushroom tyrosinase (28 nM from Sigma Chemical Co., USA) first preincubated with the compounds in 50 nM Na-phosphate buffer (pH 6.8) for 10 min at 25° C. Then, the L-DOPA (0.5 mM) was added to the reaction mixture and the enzyme reaction was monitored by measuring the change in absorbance at 475 nm (at 37° C.) due to the formation of the DOPA chrome for 10 min. The percent inhibition of the enzyme was calculated as =[B−S/S]×100, wherein the B and S are absorbance for the blank and samples, respectively. After screening, the compounds median inhibitory concentration (IC₅₀) was also calculated. All the studies have been carried out at least in triplicates and the results represent the mean±S.E.M. (standard error of the mean). Kojic acid and L-mimosine were used as standard inhibitors for the tyrosinase, and both of them were purchased from Sigma Chem. Co., USA. (Hearing et al., Int J Biochem, 19(12): 1141-71987). The concentrations of the test compounds, which inhibited 50% tyrosinase enzyme was determined and the IC₅₀ values were calculated using EZ-Fit enzyme kinetics program (Perrella Scientific inc., Amherst, Mass., U.S.A.). The hydroxy metabolites of tibolone reported above showed significant inhibitory activity against enzyme tyrosinase (Table 1).

TABLE 1
Tyrosinase inhibitory activities of tibolone metabolites as compared
to the reference inhibitors (kojic acid and L-Mimosine)

| Compounds | IC₅₀ ± SEM (in μM) |
|---|---|
| Metabolite 1 | 7.45 ± 0.21 |
| Metabolite 2 | 50.86 ± 0.36 |
| Metabolite 3 | 8.19 ± 0.54 |
| Metabolite 4 | 119.44 ± 0.20 |
| Metabolite 5 | 5.12 ± 0.67 |
| Metabolite 6 | 7.10 ± 0.13 |
| Metabolite 7 | 36.14 ± 0.21 |
| Metabolite 8 | 25.15 ± 0.24 |
| Kojic acid | 16.67 ± 0.51 |
| L-Mimosine | 3.68 ± 0.022 |

### Skin Lightening Cosmetic Composition

Skin lightening products, as well as even-toning products are becoming increasingly popular. This is true not only in the traditional Asian-Pacific and African markets, but worldwide. The main purpose of these products is to lighten, whiten, brighten, or even-tone the skin. For all skin types, these lightening agents can be used to treat pigmentation disorders, such as freckles, pregnancy masks and age spots. Skin color is mainly determined by the amount of melanin present in the skin. Melanin is synthesized in melanocytes, which are normally found in the epidermal basal layer. Within the melanocytes, melanin is bound to a protein matrix to form melanosomes. In the melanosomes, tyrosinase converts tyrosine to eumelanin or pheomelanin. By blocking at the various points of the pathways, skin lightening agents can inhibit or even reverse melanin biosynthesis and are thus useful in whitening or lightening human skin. Skin lightening agents can also be used to treat local hyperpigmentation or spots that are caused by local increase in melanin synthesis or uneven distribution.

To meet this need, many attempts have been made to develop products that reduce the pigment production in the melanocytes. However, the substances identified thus far tend to have either low efficacy or undesirable side effects, such as, for example, toxicity or skin irritation. Therefore, there is a continuing need for new cosmetic skin lightening agents, with improved overall effectiveness. For example, certain resorcinol derivatives, particularly 4-substituted resorcinol derivatives, are useful in cosmetic compositions for skin lightening benefits, as disclosed in Hu et al., U.S. Pat. No. 6,132,740, Bradley, et al, U.S. Pat. Nos. 6,504,037 and 6,861,564; Japanese published patent applications IP 2001-010925 and JP2000-327557; Harichian et al, U.S. Pat. No. 6,852,310; and Shore et al., U.S. Pat. No. 7,270,805 for the use of N-acylbenzothiazolones.

Hydroquinone is an OTC monographed drug. The Skin Lightening/Whitening Monograph is unusual in that it has only one active ingredient listed—hydroquinone. The list is unusual also in that there is no range of acceptable dosages listed. Hydroquinone is allowed only at a use level of 2.00%. A 2% hydroquinone cream is a fully legal OTC drug. It must be labeled as a drug and must be manufactured under pharmaceutical GMP. There are concerns that hydroquinone may pose a health risk. This has prompted legal debate in Korea, the Union of South Africa and elsewhere. There is concern that the U.S. and European nations may ban this material. For this reason, alternatives to hydroquinone are being studied intensely.

The following materials are actives that can be labeled as cosmetics. Products that contain them must be labeled as “skin brighteners” or “skin toners” rather than skin lighteners.

Kojic acid is a water-soluble tyrosinase inhibitor that works by competing with DOPA at its receptor site. This material can cause products containing it to discolor, causing a minor challenge to the formulation chemist. However, kojic acid has no known safety issues. Kojic acid at 1% concentration will give about the same skin-bleaching effect as hydroquinone will at 2%.

Arbutin is also a water-soluble tyrosinase inhibitor. It works in exactly the same way as kojic acid.

Magnesium Aseorbyl Phosphate (MAP) is a tyrosinase inhibitor that acts as a reducing agent on melanin intermediates. Thus, it blocks the oxidation chain reaction at various points in the transformation of tyrosinase/DOPA to melanin.

Kojic Acid Dipalmitate (KAD) KAD is a tyrosinase inhibitor. The exact mechanism of its action is unclear. This product is soluble in oil and esters. It is stable under a wide range of conditions and does not change color or discolor emulsions.

Calcium D-Pantetheine-S-Sulfonate is a water-soluble material with superior tyrosinase inhibiting affect. This patented material is stable under a wide range of conditions and has skin lightening action that is faster than that of the above-mentioned actives. Herbal Blends include Bearberry Extract which contains a high level of arbutin or a aqueous extract of Angelica dehurica roots, Cucumis saliva (cucumber) seed, Mums alba bark and Hibiscus sabdariffa flower that create a whitening effect at several levels of pigmentation, it does this by inhibiting tyrosinase activity. The presence of derived cinnamics enables it to act on the site of the enzyme by structural analogy with tyrosine, inhibiting melanin biosynthesis. Then this action is reinforced by stylbens compounds, which come from white mulberry (Moras alba bark). These act as competitive inhibitors of tyrosinase. Still another action is that of phenyalanin, obtained from the cucumber seed in the blend. This limits the membrane transfer of melanin onto the melanosoms, and consequently limits the storage of pigment and improves the whitening effect. The next level of action is linked to the organic acids of the hibiscus flower, which play an exfoliating role. Pyruvic and citric acids increase the cell turnover and the whitening of the skin is improved, while dark spots are removed. These organic acids can also act as reducing agents and allow the change of melanin into a non-pigmented form. Another blend is a hydroglycolic extract of peach, apple and raspberry. This combines the powerful anti-tyrosinase active in peach leaves with the clarifying action of raspberries and apples.

The use of compounds of the metabolism of tibolone using fermentation with fungi delivers skin lightening benefits with potential reduced irritation. The present invention provides a cosmetic composition and method of skin lightening using a composition comprising in addition to a cosmetically acceptable vehicle, about 0.000001 to about 50% of a new tibolone metabolite. Further skin benefit agents may be included in the inventive cosmetic compositions. Organic and inorganic sunscreens may also be included. The inventive compounds and compositions may be used for reducing overall skin pigmentation and the reduction of discrete hyperpigmentation, such as blemishes and freckles, as well as for reducing the irritation associated with irritating skin benefit agents, such as retinol.

As used herein, the term “cosmetic composition” is intended to describe compositions for topical application to human skin.

The term “skin” as used herein includes the skin on the face, neck, chest, back, arms, axilla, hands, legs, and scalp.

Except in the examples, or where otherwise explicitly indicated, all numbers in this description indicating amounts of material or conditions of reaction, physical properties of materials and/or use are to be understood as modified by the word “about”. All amounts are by weight of the composition, unless otherwise specified.

It should be noted that in specifying any range of concentration, any particular upper concentration can be associated with any particular lower concentration.

The term “comprising” is used herein in its ordinary meaning and means including, made up of, composed of, consisting and/or consisting essentially of. In other words, the term is defined as not being exhaustive of the steps, components, ingredients, or features to which it refers.

The invention is concerned with the use of compounds of tibolone metabolism produced by fermentation with fungi and compositions including same, as skin cosmetic agents, particularly as skin lightening agents. A particular advantage of the inventive compositions and methods is that these compounds can be less irritating to the skin than known skin lightening compounds.

Further skin benefit agents may be included in the inventive cosmetic compositions. Organic and inorganic sunscreens may also be included. The inventive cosmetic compositions and methods have effective skin lightening properties and may be less irritating to the skin.

The compositions generally contain about 0.000001 to about 50% of compounds of tibolone metabolism, preferably in the range of about 0.00001% to about 10%, more preferably about 0.001 to about 7%, most preferably from 0.01 to about 5%, of the total amount of a cosmetic composition.

The preferred cosmetic compositions are those suitable for the application to human skin according to the method of the present invention, which optionally, but preferably, include a further skin benefit agent. Suitable additional skin benefit agents include anti-aging, wrinkle-reducing, skin whitening, anti-acne, and sebum reduction agents. Examples of these include alpha-hydroxy acids, beta-hydroxy acids, polyhydroxy acids, hyaluronic acid, hydroquinone, t-butyl hydroquinone. Vitamin B derivatives. Vitamin C derivatives; allantoin, a placenta extract; dioic acids, retinoids, and resorcinol derivatives. The cosmetically acceptable vehicle may act as a dilutant, dispersant or carrier for the skin benefit ingredients in the composition, so as to facilitate their distribution when the composition is applied to the skin. The vehicle may be aqueous, anhydrous or an emulsion. Preferably, the compositions are aqueous or an emulsion, especially water-in-oil or oil-in-water emulsion, preferably oil in water emulsion. Water when present will be in amounts which may range from 5 to 99%, preferably from 20 to 70%, optimally between 40 and 70% by weight. Besides water, relatively volatile solvents may also serve as carriers within compositions of the present invention. Most preferred are monohydric C₁-C₃ alkanols, These include ethyl alcohol, methyl alcohol and isopropyl alcohol. The amount of monohydric alkanol may range from 1 to 70%, preferably from 10 to 50%, optimally between 15 to 40% by weight. Emollient materials may also serve as cosmetically acceptable carriers. These may be in the form of silicone oils and synthetic esters. Amounts of the emollients may range anywhere from 0.1 to 50%, preferably between 1 and 20% by weight. Silicone oils may be divided into the volatile and non-volatile variety. The term “volatile” as used herein refers to those materials which have a measurable vapor pressure at ambient temperature.

Volatile silicone oils are preferably chosen from cyclic or linear polydimethyl siloxanes containing from 3 to 9, preferably from 4 to 5, silicon atoms. Linear volatile silicone materials generally have viscosities less than about 5 centistokes at 25° C. while cyclic materials typically have viscosities of less than about 10 centistokes. Nonvolatile silicone oils useful as an emollient material include polyalkyl siloxanes, polyalkylaryl siloxanes and polyether siloxane copolymers. The essentially non-volatile polyalkyl siloxanes useful herein include, for example, polydimethyl siloxanes with viscosities of from about 5 to about 25 million centistokes at 25° C. Among the preferred non-volatile emollients useful in the present compositions are the polydimethyl siloxanes having viscosities from about 10 to about 400 centistokes at 25° C.

Among the ester emollients are: (1) Alkenyl or alkyl esters of fatty acids having 10 to 20 carbon atoms. Examples thereof include isoarachidyl neopentanoate, isononyl isonanonoate, oleyl myristate, oleyl stearate, and oleyl oleate. (2) Ether-esters such as fatty acid esters of ethoxylated fatty alcohols. (3) Polyhydric alcohol esters. Ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl mono-stearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters. (4) Wax esters such as beeswax, spermaceti, myristyl myristate, stearyl stearate and arachidyl behenate. (5) Sterols esters, of which cholesterol fatty acid esters are examples.

Fatty acids having from 10 to 30 carbon atoms may also be included as cosmetically acceptable carriers for compositions of this invention. Illustrative of this category are pelargonic, lauric, myristic, palmitic, stearic, isostearic, hydroxy stearic, oleic, linoleic, ricinoleic, arachidic, behenic and erucic acids.

Humectants of the polyhydric alcohol-type may also be employed as cosmetically acceptable carriers in compositions of this invention. The humectant aids in increasing the effectiveness of the emollient, reduces scaling, stimulates removal of built-up scale and improves skin feel. Typical polyhydric alcohols include glycerol, polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, 1,2,6-hexanetriol, ethoxylated glycerol, propoxylated glycerol and mixtures thereof. For best results the humectant is preferably propylene glycol or sodium hyaluronate. The amount of humectant may range anywhere from 0.5 to 30%, preferably between 1 and 15% by weight of the composition.

Thickeners may also be utilized as part of the cosmetically acceptable carrier of compositions according to the present invention. Typical thickeners include crosslinked acrylates (e.g., Carbopol 982), hydrophobically-modified acrylates (e.g. Carbopol 1382), cellulosic derivatives and natural gums. Among useful cellulosic derivatives are sodium carboxy methyl cellulose, Hydroxypropyl methylcellulose, hydroxypropyl cellulose, hydroxyethyl cellulose, ethyl cellulose and hydroxymethyl cellulose. Natural gums suitable for the present invention include guar, xanthan, sclerotium, carrageenan, pectin and combinations of these gums. Amounts of the thickener may range from 0.0001 to 5%, usually from 0.001 to 1%, optimally from 0.01 to 0.5% by weight.

Collectively the water, solvents, silicones, esters, fatty acids, humectants and/or thickeners will constitute the cosmetically acceptable carrier in amounts from 1 to 99.9%, preferably from 80 to 99% by weight.

An oil or oily material may be present, together with an emulsifier to provide either a water-in-oil emulsion or an oil-in-water emulsion, depending largely on the average hydrophilic-lipophilic balance (HLB) of the emulsifier employed.

Surfactants may also be present in cosmetic compositions of the present invention. Total concentration of the surfactant will range from 0.1 to 40%, preferably from 1 to 20%, optimally from 1 to 5% by weight of the composition. The surfactant may be selected from the group consisting of anionic, nonionic, cationic and amphoteric actives.

Particularly preferred nonionic surfactants are those with a C.sub.10-C.sub.20 fatty alcohol or acid hydrophobe condensed with from 2 to 100 moles of ethylene oxide or propylene oxide per mole of hydrophobe; C₂-C₁₀ alkyl phenols condensed with from 2 to 20 moles of alkylene oxide; mono- and di-fatty acid esters of ethylene glycol; fatty acid monoglyceride; sorbitan, mono- and di-C₈-C₂₀ fatty acids; block copolymers (ethylene oxide/propylene oxide); and polyoxyethylene sorbitan as well as combinations thereof. Alkyl poly glycosides and saccharide fatty amides (e.g. methyl gluconamides) are also suitable nonionic surfactants.

Preferred anionic surfactants include soap, alkyl ether sulfate and sulfonates, alkyl sulfates and sulfonates, alkylbenzene sulfonates, alkyl and dialkyl sulfosuccinates, C₈-C₂₀ acyl isethionates, acyl glutamates, C₈-C₂₀ alkyl ether phosphates and combinations thereof.

Other adjunct minor components may also be incorporated into the cosmetic compositions. These ingredients may include coloring agents and/or pigments; opacifiers, perfumes, other thickeners, plasticizers; calamine; antioxidants; chelating agents, as well as additional sunscreens, such as organic sunscreens. Amounts of these other adjunct minor components may range anywhere from 0.001% up to 20% by weight of the composition.

For use as sunscreen, metal oxides may be used alone or in mixture and/or in combination with organic sunscreens. Examples of organic sunscreens include but are not limited to include: Benzophenones, UV-24 Methoxycinnamate, Ethyl dihydroxypropyl, PABA, Glyceryl PABA, Homosalate, Methyl anthranilate, Octocrylene, Octyl dimethyl PABA, Octyl methoxycinnamate, Octyl salicylate, 2-Phenylbenzimidazole-5, sulphonic add TEA salicylate, 3-(4-methylbenzylidene), 4-Isopropyl dibenzoyl, Butyl methoxy dibenzoyl, Etocrylene. The amount of the organic sunscreens in the cosmetic composition is preferably in the range of about 0.1 wt % to about 10 wt %, more preferably about 1 wt % to 5 wt %. Preferred organic sunscreens are PARSOL MCX and Parsol 1789, due to their effectiveness and commercial availability.

The method according to the invention is intended primarily as using a personal care product for topical application to human skin, for cosmetic benefits including but not limited to skin lightening. The inventive compounds and compositions may be used for reducing overall skin pigmentation and the reduction of discrete hyperpigmentation, such as blemishes and freckles, as well as for reducing the irritation associated with irritating skin benefit agents, such as retinol.

In use, a small quantity of the composition, for example from 1 to 5 ml, is applied to areas of the skin, from a suitable container or applicator and, if necessary, it is then spread over and/or rubbed into the skin using the hand or fingers or a suitable device. The cosmetic composition useful for the method of the invention can be formulated as a lotion having a viscosity of from 4,000 to 10,000 mPas, a fluid cream having a viscosity of from 10,000 to 20,000 mPas or a cream having a viscosity of from 20,000 to 100,000 mPas, or above. The composition can be packaged in a suitable container to suit its viscosity and intended use by the consumer. For example, a lotion or fluid cream can be packaged in a bottle or a roll-ball applicator or a propellant-driven aerosol device or a container fitted with a pump suitable for finger operation. When the composition is a cream. It can simply be stored in a non-deformable bottle or squeeze container, such as a tube or a lidded jar. When the composition is a solid or semi-solid stick, it may be packaged in a suitable container for manually or mechanically pushing out or extruding the composition. The invention accordingly also provides a closed container containing a cosmetically acceptable composition as herein defined.

### Enzymatic Browning Inhibition

Appearance, flavor, texture and nutritional value are four attributes considered by consumers when making food choices. Appearance which is significantly impacted by color is one of the first attributes used by consumers in evaluating food quality. Color may be influenced by naturally occurring pigments such as chlorophylls, carotenoids and anthocyanins in food, or by pigments resulting from both enzymatic and non-enzymatic reactions. Enzymatic browning is one of the most important color reactions that affect fruits, vegetables and seafood. It is catalysed by the enzyme polyphenol oxidase (1,2-benzenediol; oxygen oxidoreductase, EC1.10.3.1) which is also referred to as phenoloxidase, phenolase, monophenol oxidase, diphenol oxidase and tyrosinase. (Enzymatic Browning in Fruits, Vegetables and Seafoods, Maurice R. Marshall, jeongmok Kim and Cheng-I Wei, FAO: http://www.fao.org/ag/ags/agsi/ENZYMEFINAL/Enzymatic%20Browning.html; Parvez S, Kang M, Chung H S, Bae H, Naturally occurring tyrosinase inhibitors: mechanism and applications in skin health, cosmetics and agriculture industries. Phytother Res. 2007 September; 21(9):805-16).

Enzymatic browning is one of the most studied reactions in fruits, vegetables and seafood. Researchers in the fields of food science, horticulture, plant and postharvest physiology, microbiology, and even insect and crustacean physiology have studied this reaction because of the diversity of its impact in these systems. Polyphenol oxidases are responsible for development, of the characteristic golden brown color in dried fruits such as raisins, prunes, dates and figs. Blanching is generally required for inactivation of the enzyme after color development, in order to minimize discoloration. Polyphenol oxidases are believed to play key physiological roles both in preventing insects and microorganisms from attacking plants and as part of the wound response of plants and plant products to insects, microorganisms and bruising. As fruits and vegetables ripen, their susceptibility to disease and infestation is increased due to a decline in their phenolic content. Phenoloxidase enzymes endogenous to fruits and vegetables catalyze the production of quinones from their phenolic constituents. Once formed, these quinones undergo polymerization reactions, leading to the production of melanins, which exhibit both antibacterial and antifungal activity and assist in keeping the fruit and/or vegetable physiologically wholesome.

Increases in fruit and vegetable markets projected for the future will not occur if enzymatic browning is not understood and controlled. Enzymatic browning is one of the most devastating reactions for many exotic fruits and vegetables, in particular tropical and subtropical varieties. It is estimated that over 50 percent losses in fruit occur as a result of enzymatic browning. Such losses have prompted considerable interest in understanding and controlling phenoloxidase enzymes in foods. Lettuce, other green leafy vegetables, potatoes and other starchy staples, such as sweet potato, breadfruit, yam, mushrooms, apples, avocados, bananas, grapes, peaches, and a variety of other tropical and subtropical fruits and vegetables, are susceptible to browning and therefore cause economic losses for the agriculturist. These losses are greater if browning occurs closer to the consumer in the processing scheme, due to storage and handling costs prior to this point. The control of browning from harvest to consumer is therefore very critical for minimizing losses and maintaining economic value to the agriculturist and food processor. Browning can also adversely affect flavor and nutritional value.

Aquatic organisms rely on polyphenol oxidases to impart important physiological functions for their development. Polyphenol oxidases are important in hardening of the shell (sclerotization), after molting in insects and in crustaceans such as shrimp and lobsters. Polyphenol oxidase is also responsible for wound healing. The mechanism of wound healing in aquatic organisms is similar to that which occurs in plants in that the compounds produced as a result of the polymerization of quinones exhibit both antibacterial and antifungal activities. Unfortunately, polyphenoloxidase-catalysed browning of the shell postharvest, adversely affects both the quality and consumer acceptability of these products.

Browning or melanosis in aquatic foods postharvest occurs primarily in crustaceans. These highly prized and economically valuable products are extremely vulnerable to enzymatic browning. Melanosis is usually more severe in lobsters if the head is retained during storage postharvest. If the head is removed, care should be taken to thoroughly wash the tail in order to eliminate proteases that activate latent polyphenol oxidases and promote browning. Although the products of melanosis are not harmful and do not influence flavor or aroma, consumers will not select these products since their brown discoloration connotes spoilage. Severe melanosis on these products can cause tremendous economic losses due to the high value commanded by these aquatic products in the marketplace. There are many examples of imported aquatic products entering the United States, worth millions of dollars that are reduced markedly or lost completely owing to the severity of melanosis. Unfortunately, a majority of these products originate in developing countries, which lack both the scientific and technical resources, and the processing infrastructure required in order to prevent the occurrence of these devastating losses. Limited susceptibility of a number of crustacean species to melanosis on the other hand, presents the processor with the problem of deciding how to treat the product in order to prevent melanosis.

On the basis of the foregoing discussions, it is clear that browning has both beneficial and deteriorative effects. Control of the deteriorative effects of browning therefore poses a major challenge to the food scientist The control of browning in fruits and vegetables hinges upon an understanding of the mechanism(s) responsible for browning in fruits, vegetables and seafood, the properties of polyphenol oxidase enzyme(s), their substrates and inhibitors, and the chemical, biological and physical factors which affect, each of these parameters. Once understood these mechanisms may be applied in either preventing the browning reaction, or slowing its rate, thus extending the shelf life of the product.

Enzymatic browning does not occur in intact plant cells since phenolic compounds in cell vacuoles are separated from the polyphenol oxidase which is present in the cytoplasm. Once tissue is damaged by slicing, cutting or pulping, however, the formation of brown pigments occurs. Both the organoleptic and biochemical characteristics of fruits and vegetables are altered by pigment formation. The rate of enzymatic browning in fruit and vegetables is governed by the active polyphenol oxidase content of the tissues, the phenolic content of the tissue, pH and temperature and oxygen availability within the tissue.

As polyphenol oxidase catalyses the oxidation of phenols to o-quinones, which are highly reactive compounds. O-quinones thus formed undergo spontaneous polymerization to produce high-molecular-weight compounds or brown pigments (melanins). These melanins may in turn react with amino acids and proteins leading to enhancement of the brown color produced. Many studies have focused on either inhibiting or preventing polyphenol oxidase activity in foods. Various techniques and mechanisms have been developed over the years for the control of these undesirable enzyme activities. These techniques attempt to eliminate one or more of the essential components (oxygen, enzyme, copper, or substrate) from the reaction.

i) The elimination of oxygen from the cut surface of fruits or vegetables greatly retards the browning reaction. Browning however occurs rapidly upon exposure to oxygen. Exclusion of oxygen is possible by immersion in water, syrup, brine, or by vacuum treatment.
ii) This copper prosthetic group of polyphenol oxidases must be present for the enzymatic browning reaction to occur. Chelating agents are effective in removing copper.
iii) Inactivation of the polyphenol oxidases by heat treatments such as steam blanching is effectively applied for the control of browning in fruits and vegetables to be canned or frozen. Heat treatments are not however practically applicable in the storage of fresh produce.
iv) Polyphenol oxidase catalyses the oxidation of phenolic substrates such as caffeic acid, protocatechuic acid, chlorogenic acid, and tyrosine. Chemical modification of these substrates can however prevent oxidation. Thus the use of tyrosine inhibitors plays a significant role in controlling the browning reaction kojic acid inhibits the rate of formation of pigmented products, as well as the rate of oxygen uptake, when various o-dihydroxy- and trihydroxy phenols are oxidized by tyrosinase. Tyrosinase inhibition by kojic acid was thought to be due to the ability of kojic acid to bind copper at the active site of the enzyme. Although kojic acid is a good inhibitor of polyphenol, oxidase, its toxicity is of concern since studies have shown weak mutagenic activity of kojic acid in a Salmonella typhimurium assay. It is therefore important to find better alternates to kojic acid.
v) Certain chemical compounds react with the products of polyphenol oxidase activity and inhibit the formation of the colored compounds produced in the secondary, non-enzymatic reaction steps, which lead to the formation of melanin.

In this invention, we report novel tyrosinase inhibitors that can be used to prevent enzymatic browning.

### REFERENCES

1. Choudhary, M. I.; Nasir, M; Khan, S. N.; Axifi M.; Ali, R. A.; Khalil, S. M; Attaur-Rahman Z. Naturforsch. 2007, 62b, 593-599.
2. Choudhary, M, I.; Yousuf, S.; Samreen.; Shah, S. A. A.; Ahmed, S.; Atta-ur-Rahman Chem. Pharma. Bull. 2006, 54, 927-930.
3, Choudhary, M. I; Shah, S. A. A. Sami, A.; Ajaz, A.; Shaheen, F.; Atta-ur-Rahman Chem. Biodiv. 2005, 2, 516-524.
4. Choudhary, M. I.; Batool, I.; Shah, S. A. A.; Nawaz, S. A.; Atta-ur-Rahman Chem. Pharma. Bull. 2005, 53, 1455-1459.
5. Choudhary, M. I.; Sultan, S.; Jalil, S.; Anjum, S.; Rahman, A. A.; Fun, H. K.; Atta-ur-Rahman Chem. Biodiv. 2005, 2, 392-400.
6. Da-Fang, Z.; Lu, S.; Lei, L.; Hai-Hua, H. Acta. Pharmacol. 2003, 24, 442-447.
7. Moody, J. D.; Zhang, D.; Heinze, T. M.; Cerniglia, C. E. Appl. Envirn. Microbiol. 2000, 66, 3646-3649.
8. Abourashed, E. A.; Clark, A. M.; Hufford, C. D. Curr. Med. Chem. 1999, 6, 359-374.
9. Chatterjee, P.; Kauzi, S. A.; Pezzuto, J. M.; Hamann, M. T. Appl. Envirn. Microbiol. 2000, 66, 3850-3855.
10. Kent, U. M; Jushchyshyn, M. I; Hollenberg, P. F. Curr. Drug. Meta. 2001, 2, 215-243.
11. Hu, S. H.; Tian, X. F.; Sun Y. R; Han, G. D. Steroids 1996, 61, 407-410.
12. Hu, S. H; Tian, X. F.; Han, G. D. Steroids 1998, 63, 88-92.
13. Choudhary, M, I.; Musharraf, S. G.; Ali, R. A.; Atif, M.; Atta-ur-Rahman Z. Naturforsch. 2004, 59b, 323-328.
14. Ederveen, A, J.; Kloosterboer, H. J. J. Bone Miner. Res. 1999, 14, 1963-1970.
15. Kloosterboer, H. J. J. Steroid Biochem. Mol. Biol. 2001, 76, 231-238.
16. Vos, R. M. E.; Krebbers, S. F. M.; Verhoever, C. H. J.; Delbressine, L. P. C. Drug. Metabol. disposi. 2002, 30, 106-112.
17. Steckelbroeek, S.; Jin Y.; Oyesanni, B.; Kloosterboer, H. J.; Penning, T. M. Mol. Pharmacol. 2004, 66, 1702-1711.
18. Helenius, J.; Kloosterboer, H. J. Maturitas, 2004, 48, 30-40
19. Yazigi, R.; Sahid, S.; Contreras, L.; Rodriguez, T. Gyneco. Onco. 2004, 93, 568-570.
20. Morin-Papunen, L. C.; Vauhkonen, L.; Ruokonen, A.; Tapanainen, J. S.; Raudaskoski, T. Eur. J. Endocrinol. 2004, 150, 705-714.
21. Choudhary, M. I.; Musharraf, S. G.; Khan, M. T. H.; Aldelrahman, D.; Parvaz, M.; Shaheen, F.; Atta-ur-Rahman Helv. Chim. Acta. 2003, 86, 3450-3460.
22. Choudhary, M. I.; Shah, S. A. A.; Musharraf, S. G.; Shaheen, F.; Atta-ur-Rahman Nat. Prod. Res. 2003, 17, 215-220.
23. Choudhary, M. I.; Musharraf, S. G.; Shaheen, F.; Atta-ur-Rahman Nat. Prod. Lett. 2002, 16, 377-382.
24. Choudhary, M. I.; Azizuddin.; Atta-ur-Rahman Nat. Prod. Lett. 2001, 16, 101-106.
25. Atta-ur-Rahman, Choudhary, M. I.; Asif, F.; Farooq, A.; Yaqoob, M. Nat. Prod. Lett. 2000, 217-224.
26. Atta-ur-Rahman, Choudhary, M. I.; Asif, F.; Farooq, A.; Yaqoob, M.; Dar, A. Phytochemistry 1998, 49, 2341-2342.
27. Atta-ur-Rahman, Yaqoob, M.; Farooq, A.; Anjum, S.; Asif, F.; Choudhary, M. I. J. Nat. Prod 1998, 61, 1340-1342.
28. Atta-ur-Rahman, Choudhary, M. I; Shaheen, F.; Ashraf, M.; Jahan, S. J. Nat. Prod. 1998, 67, 428-431.
29. Atta-ur-Rahman, Farooq, A.; Anjum, S.; Choudhary, M. I. Curr. Org. Chem. 1999, 3, 309-312.
30. Atta-ur-Rahman, Farooq, A.; Choudhary, M. I. J. Nat. Prod. 1997, 60, 1038-1040.
31. Chiba, S. Biosci. Biotech. Biochem. 1997, 61, 1233-1239.
32. Crespo, C. J.; Smit, E.; Snelling A.; Sempos, C. T.; Andersen, R. E. Diabetes Care 2002, 25, 1675-1680.
33. Egarter, C.; Huber, J.; Haidbauer, R.; Pusch, H.; Fischl, F.; Putz, M. Maturitas 1996, 23, 55-62.
34. Castelo-Branco, C.; Vicente, J. J.; Figueras, F.; Sanjuan, A.; deOsaba, M. J. M; Casals, E.; Pons, F.; Balasch, J.; Vanrell, J. A. Maturitas 2000, 32, 161-168.
35. Kamel, H. K.; Perry, H. M; Motley, J. E. J. Am. Geriatr. Soc. 2001, 49, 179-187.
36. Iqbal, M. M. South Med. J. 2000, 93, 2-18.
37. Lindsay, R.; Cosman, F.; Lobo, R. A.; Walsh, B. W.; Harris, S. T.; Reagan, J. E.; Liss, C. L.; Melton, M. E.; Byrnes C. A. J. Clin. Endocrinol. Metab. 1999, 84, 3076-3081.
38. Shiino, M.; Watanabe, Y.; Umezawa, K. Bioorg. Med. Chem. 2001, 9, 1233-1240.
39. Hanson, J. R.; Nasir, H.; Parvez, A. Phytochemistry 1996, 42, 411-415.
40. Lee, P.; Kitamura, Y.; Kaneko, K.; Shiro, M.; Xu, G. J.; Chen, Y. P. Chem. Pharma. Bull. 1988, 36, 4316-4329.
41. Hearing, V. J. Methods Enzymol. 1987, 142, 154-165.
42. Matsui, T.; Yoshimoto, C.; Osajima, K.; Oki, T.; Osajima, Y. Biosci. Biotech. Biochem. 1996, 60, 2010.

What is claimed is:

1. A new chemical compound selected from a group consisting of 6β-Hydroxytibolone, 15β-Hydroxytibolone, Δ^{1,4}-Tibolone, 11α,15β-Dihydroxytibolone, 11α,15β-Dihydroxy-Δ⁵-tibolone, 6β-Hydroxy-Δ⁴-tibolone, 6β-Methoxy-Δ⁴-tibolone, and 6β-Hydroxytibolone, its derivatives, isomers and salts thereof.

2. A method of inhibiting tyrosinase enzyme by contacting with Δ⁴-Tibolone, 6β-Hydroxytibolone, 15β-Hydroxytibolone, Δ^{1,4}-Tibolone, 11α,15β-Dihydroxytibolone, 11α,15β-Dihydroxy-Δ⁵-tibolone, 6β-Hydroxy-Δ⁴-tibolone, and 6β-Methoxy-Δ⁴-tibolone.

3. A method of producing 6β-Hydroxytibolone, 15β-Hydroxytibolone, and Δ⁴-Tibolone by contacting tibolone with Rhizopus stolonifer (TSY 0471) under suitable fermentation conditions.

4. A method of producing Δ^{1,4}-Tibolone, 11α,15β-Dihydroxytibolone, and 11α,15β-Dihydroxy-Δ⁵-tibolone by contacting tibolone with Cunninghamella elegans (TSY 0865) under suitable fermentation conditions.

5. A method of producing 6β-Hydroxy-Δ⁴-tibolone and 6β-Methoxy-Δ⁴-tibolone by contacting tibolone with Gibberella fujikuroi (ATCC 10704) under suitable fermentation conditions.

6. A cosmetic method of skin lightening comprising applying to the skin a composition comprising about 0.000001 to about 50% of a compound of claim 2 and a cosmetically acceptable carrier.

7. The method of claim 6, wherein said composition further comprises a sunscreen.

8. The method of claim 6, wherein said sunscreen is a micronized metal oxide.

9. The cosmetic method according to claim 6, wherein said composition further comprises a skin benefit agent selected from the group consisting of alpha-hydroxy acids, beta-hydroxy acids, polyhydroxy acids, betulinic acid, hydroquinone, t-butyl hydroquinone, Vitamin C derivatives, dioic acids, retinoids, resorcinol derivatives, and mixtures thereof.

10. The cosmetic method of claim 6, wherein the said composition further comprises an organic sunscreen selected from the group consisting of Benzophenone-3, Benzophenone-4, Benzophenone-8, DEA, Methoxycinnamate, Ethyl dihydroxypropyl-PABA, Glyceryl PABA, Homosalate, Methyl anthranilate, Octocrylene, Octyl dimethyl PABA, Octyl methoxycinnamate (PARSOL MCX), Octyl salicylate, PABA, 2-Phenylbenzimidazole-5-sulphonic acid, TEA salicylate, 3-(4-methylbenzylidene)-camphor, Benzophenone-1, Benzophenone-2, Benzophenone-6, Benzophenone-12, 4-Isopropyl dibenzoyl methane, Butyl methoxy dibenzoyl methane (PARSOL 1789), Etocrylene, and mixtures thereof.

11. The cosmetic method of claim 6, wherein the said composition further comprises skin lightening agents hydroquinone, kojic acid, arbutrin, magnesium ascorbyl phosphate, kojic acid dipalmitate, calcium D-pantetheine-S-sulfonate, blue berry extract, extract of Angelica dehurica roots, Cucumis sativa (cucumber) seed, Murus alba bark or Hibiscus sabdariffa flower.

12. A method of preserving food products against enzymatic browning by contacting the food products, fruits, vegetables, meats, with an effective amount of the said tyrosinase inhibitor of claim 2, optionally contained in a suitable carrier.

13. As claimed in claim 12, wherein the said method involves combining the application with known agents that prevent enzymatic browning.
